(19) United States
(12) Patent Application Publication
(10) Pub. No.: US 2011/0071622 A1
(43) Pub. Date: Mar. 24, 2011

(54) INSTRUMENT FOR THE SURGICAL TREATMENT OF AORTIC VALVE DEFECTS

(75) Inventors: Guido FEHLING, Karlstein (DE); Boulos ASFOUR, Bonn (DE)

(73) Assignee: FEHLING INSTRUMENTS GMBH & CO. KG, Karlstein (DE)

(21) Appl. No.: 12/889,152

(22) Filed: Sep. 23, 2010

(30) Foreign Application Priority Data
Sep. 23, 2009 (DE) 10 2009 042 465.2

(51) Int. Cl. A61F 2/24

(52) U.S. Cl. 623/2.1

(57) ABSTRACT

The invention relates to an instrument for the surgical treatment of aortic valve defects, in particular in young patients such as children, consisting of a shaft, a handle arranged at the proximal end of the shaft, and a leaflet body which is arranged at the distal end of the shaft and which has substantially the shape of the sinus of the aortic valve leaflet.

### CROSS REFERENCE TO RELATED APPLICATIONS

This application is a continuation of German Patent Application No. DE 10 2009 042 465.2 filed Sep. 23, 2009, which is hereby incorporated by reference in it's entirety.

### FIELD OF INVENTION

The invention relates to an instrument for the surgical treatment of aortic valve defects, in particular in young patients such as children.

### BACKGROUND

The aortic valve (valva aortae) is arranged as valve system in the aortic opening of the left ventricle which opens during the systole under ventricular pressure of the blood and closes during the diastole under arterial pressure. The aortic valve has three valve leaflets which are attached via commissures at the annulus fibrosus. The valve leaflets have semilunar valve cusps (valvulae semilunares) which form a valve cusp sinus (sinus valsalvae). The valve cusps abut against each other with their free leaflet edges in the closed position in a sealing manner to prevent a back current of the blood from the aorta into the left ventricle. In the open position, the free edges of the elastic flexible cusps are moved away from each another.

Aortic valve defects can result in an aortic valve insufficiency or aortic valve stenosis. In case of the aortic valve insufficiency, the artic valve does not close completely whereas in case of the aortic valve stenosis, the opening of the aortic valve is narrowed. Different surgical methods for the treatment of aortic valve defects are known. The defect aortic valve can be completely or partially replaced by a mechanical or biological valve prosthesis. In addition, surgical methods are known in which the patient's own aortic valve remains intact to the greatest possible extent and the function is restored by surgical reconstruction. Typical here are in particular plicatures or tightening the cusps or reconstructions of the connection of the aortic valve. Valve prostheses involve the risk of rejections and thromboses. Therefore, a reconstruction of the aortic valve is to be preferred if this is possible.

Aortic valve defects in children, which are usually congenital, involve primarily aortic valve insufficiencies wherein the valve leaflets are malformed or not completely developed. In such patients, the reconstruction of the aortic valve is of particular importance to allow the aortic valve to grow along and to avoid a potentially lifelong medication.

### SUMMARY

The invention is based on the object to facilitate and improve the surgical treatment of aortic valve defects, in particular in young patients such as children.

This object is solved according to an instrument for the surgical treatment of aortic valve defects, in particular in young patients such as children, having the structures and features recited herein. Advantageous embodiments of the invention are specified in the sub-claims.

According to the invention, the surgeon is provided with an instrument which makes the surgical intervention easier and more reliable and by means of which a defect aortic valve can be converted into a functionally sufficient aortic valve. In case of acquired aortic valve insufficiencies, the natural aortic valve with its functional capability can be reconstructed. The instrument is in particular suitable for the correction of congenital aortic valve insufficiencies, wherein formally and functionally sufficient valve leaflets are made from the existing defect aortic valve leaflets.

The instrument according to the invention consists of a shaft having a proximal handle and a leaflet body arranged at the distal shaft end. The leaflet body has substantially the shape of the inner sinus of the valve leaflet. The instrument can be used in two ways. First, the instrument can be used as gage to palpate the valve leaflet. For this, a set of instruments each with differently sized leaflet bodies is used. By inserting the different leaflet bodies into a valve leaflet having a damage as small as possible, the surgeon can determine the optimal size of the valve leaflets. Subsequently, the instrument with the optimal leaflet body determined through the gaging is used as shaping body, around which the surgeon constructs a formally and functionally sufficient valve leaflet using the body's own valve tissue or pericardial tissue. The leaflet body of the instrument is used here in a similar manner as the last for manufacturing shoes. By shaping and suturing the tissue around the shaping leaflet body it is made easier for the surgeon to operatively construct a valve leaflet which is almost identical to the geometrical shape of the natural valve leaflet.

To optimize the handling of the instrument, the shaft of the instrument is flexible and in particular plastically deformable. The surgeon can bend the shaft for an optimal handling so as to insert the leaflet body in the anatomically most advantageous way and under the most advantageous visibility and surgery conditions into the respective valve leaflet.

In an advantageous embodiment, the shaft is made of a memory metal, e.g. a martensitic nickel-titanium alloy which is plastically deformable at room temperature and which adopts its original shape again upon exceeding the higher upper transition temperature. The instrument is available with a straight shaft and can be bent by the surgeon into the suitable shape for the subsequent use. The shaft maintains this shape until the transition temperature is exceeded during the instrument preparation in the washing machine and in the autoclave and the shaft of the instrument adopts its straight original shape again.

### BRIEF DESCRIPTION OF DRAWINGS

Hereinafter, the invention is explained in more detail by means of an exemplary embodiment illustrated in the drawing. In the figures:

FIG. 1 shows a schematic illustration of the aortic valve in a top view of the aorta,

FIG. 2 shows schematically an individual valve leaflet,

FIG. 3 shows an axial section through the valve leaflet,

FIG. 4 shows an overall view of the instrument according to the invention, and

FIGS. 5a, b and c show the leaflet body of the instrument in different views.

### DETAILED DESCRIPTION

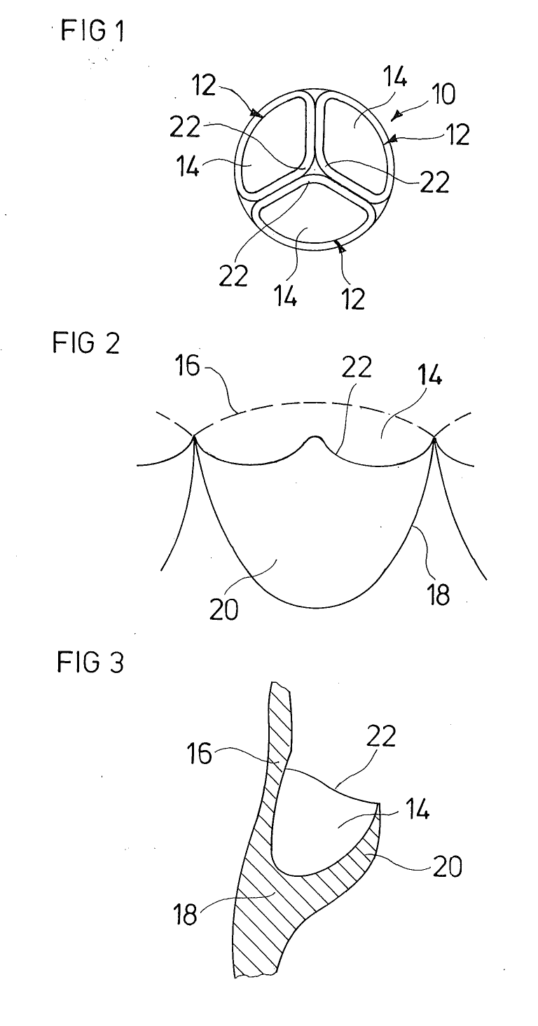

The anatomical structure and shape of an aortic valve (valva aortae) is schematically illustrated in the FIGS. 1 to 3.

The aortic valve 10 has three valve leaflets 12. They fill the cross-section of the aortic valve 10 substantially in the form of three sectors of a circle as it is shown in FIG. 1. Each valve leaflet 12 has the shape of a pocket, the inner pocket volume of which is designated as sinus valsalvae 14 and is open distal to the aorta. The outer wall of the valve leaflets 12 extends from the sinotubular junction 16 as the upper border line up to the annulus fibrosus 18. Three valve cusps 20, which have an extremely elastic, flexible structure, extend from the annulus fibrosus 18. With the valves closed, the cusps 20 abut against each other with their free edges in a triangle shape and in sealing manner, wherein the arterial pressure in the aorta fills the sinus valsalvae 14 and the valve cusps 20 abut against each other with their free edges 22. In the systole, the aortic valve opens, wherein the valve cusps 20 move radially away from each other due to their elastic tissue structure and open a passage cross-section between their free edges 22 for the systolic bloodstream.

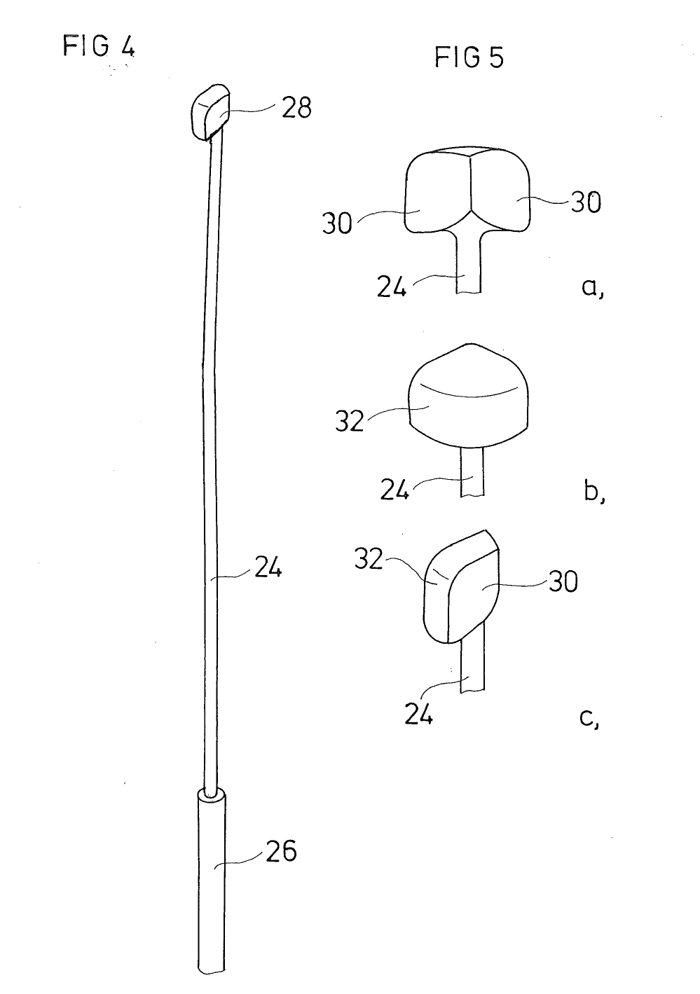

The instrument illustrated in an exemplary embodiment in the FIGS. 4 and 5 has an elongated thin shaft 24 at the proximal end of which, for example, a cylindrical handle 26 is attached. At the distal end of the shaft 24, a leaflet body 28 is attached.

The shaft 24 is preferably made of a memory metal, in particular a martensitic nickel-titanium alloy. In its original shape, the shaft 24 extends in a straight line. Due to the memory properties of the material, the shaft 24 is plastically deformable at room temperature and can be bent in any desired curvature shape. The shaft 24 maintains said curvature shape at room temperature. If the shaft 24 is heated above its upper transition temperature, which lies above the room temperature, e.g. during the instrument preparation in a washing machine or in the autoclave, it adopts its straight original shape again.

The leaflet body 28 has substantially the shape of the sinus 14 of the valve leaflets 12. Accordingly, the leaflet body 28 has two flat surfaces 30 which run substantially at an angle of 120° to each other and a circular-cylindrically curved outer surface 32. The cross-section of the leaflet body 28 thus corresponds substantially to the cross-section of the sinus 14 of the valve leaflet 12 in the closed position of the valves as it is shown in FIG. 1. The axial dimension (in the axial direction of the shaft 24) of the leaflet body 28 corresponds substantially to the axial depth of the sinus 14, wherein the distal end of the leaflet body 28 is rounded towards the outer surface 32 as it corresponds to the anatomic shape of the sinus 14.

The instrument 28 is produced with differently sized leaflet bodies 28 which correspond to the different sizes of the aortic valve 10, in particular in young patients such as children.

For a surgical operation of an aortic valve defect, in particular in young patients such as children, the surgeon selects first the aortic valve's 10 valve leaflet 12 with the best shape and gages the sinus 14 of the same. For this, he uses successively a plurality of instruments with differently sized leaflet bodies 28 to determine the size of the leaflet body 28 which corresponds best to the valve leaflet 12.

The defect or malformed valve leaflets 12 are then surgically constructed by using the selected leaflet body 28 as shaping body and the malformed or missing valve cusp 20 is formed around said leaflet body 28. For this, the surgeon uses the defect valve tissue or, if this is not sufficient, pericardial tissue can also be used. The tissue is formed around the leaflet body 28 and sutured. If necessary, the other valve leaflets 12 of the aortic valve 10 are constructed in the same manner.

By means of the instrument according to the invention, the surgeon can construct, in a relatively simple and reliable manner, geometrically identical valve leaflets 12 which come as close as possible to the natural shape of the valve leaflets 12. Since the valve leaflets are made of the patient's own body tissue, there are no problems with rejections and no risk of thrombosis.

### REFERENCE NUMBER LIST

10 Aortic valve

12 Valve leaflets

14 Sinus valsalvae

16 Sinotubular junction

18 Annulus fibrosus

20 Valve cusp

22 Free edge

24 Shaft

26 Handle

28 Leaflet body

30 Flat surfaces

32 Outer surface

What is claimed:

1. An instrument for the surgical treatment of aortic valve defects, comprising:
a shaft having a proximal and distal end;
a handle arranged at the proximal end of the shaft; and
a leaflet body which is arranged at the distal end of the shaft and which has substantially the shape of a sinus of an aortic valve leaflet.

2. The instrument according to claim 1, wherein the shaft is bendable.

3. The instrument according to claim 2, wherein that the shaft can be plastically deformed.

4. The instrument according to claim 3, wherein the shaft is made of a memory metal with a straight original shape, wherein the memory metal can be plastically deformed at room temperature and, upon exceeding an upper transition temperature, adopts its straight original shape again.

5. The instrument according to claim 1, wherein the instrument is available with different sizes of the leaflet body.

6. The instrument according to claim 1, wherein the leaflet body comprises two flat surfaces which run substantially at an angle of 120° to each other and a circular cylindrically curved outer surface.

7. The instrument according to claim 4, wherein the memory metal is nickel-titanium alloy.
